(19) United States
(12) Patent Application Publication
(10) Pub. No.: US 2016/0174844 A1
(43) Pub. Date: Jun. 23, 2016

(54) DENTAL TREATMENT IMPLEMENT WITH ANOMALY SITE DETECTION FUNCTION

(71) Applicant: THE YOSHIDA DENTAL MFG. CO., LTD., Tokyo (JP)

(72) Inventors: Takeshi HASEGAWA, Tokyo (JP); Michizo YAMANAKA, Tokyo (JP)

(21) Appl. No.: 14/909,647

(22) PCT Filed: Aug. 1, 2014
(86) PCT No.: PCT/JP2014/070314

§ 371 Date: Feb. 2, 2016

(30) Foreign Application Priority Data
Aug. 2, 2013 (JP) 2013-161416

(51) Int. Cl. A61B 5/00; A61C 1/06; A61B 1/06; A61C 1/12; A61C 3/02; A61C 1/05; A61C 1/10

(52) U.S. Cl. CPC A61B 5/0071; A61B 5/0088; A61C 1/05; A61C 1/06; A61C 1/10; A61C 1/12; A61C 3/02; A61B 1/0646

(57) ABSTRACT

[Problem] The present invention provides a dental treatment implement with an anomaly site detection function, capable of accurately detecting an anomaly site on a tooth and gingiva (marginal part) in an oral cavity using an image, accurately treating the site on the basis of the image, and performing MI and PMTC in an efficient and reliable manner.

[Solution] The present invention includes: a micromotor handpiece 1 with an anomaly site detection function, the micromotor handpiece 1 being a dental treatment implement for treating an anomaly site in an oral cavity: a color camera module group 21 including a plurality of color camera modules 21A, 21B, and 21C, wherein each color camera module is constructed by integrating a light emission means including a light source unit 21A, 27B, or 27C for at least emitting excitation light for producing fluorescent light at an anomaly site in the oral cavity and an image pick-up means for picking up an image of the anomaly site and surrounding sites in the oral cavity as a color image, wherein the wavelengths of the excitation light emitted from the light source units 27A, 27B, and 27C are different from each other, and wherein, one of the color camera modules is detachably mounted in a specific position on the micromotor handpiece 1 with the anomaly site detection function; and a light emission/image pick-up optical system 10 for radiating the excitation light from the light source unit 27A, 27B; or 27C of the color camera module 21A, 21B, or 21C mounted in the specific position coward the anomaly site and guiding the fluorescent light produced at the anomaly site to a camera unit 31 of the color camera module 21A, 21B, or 21C.

### TECHNICAL FIELD

The present invention relates to a dental treatment implement with an anomaly site detection function, more specifically, to a dental treatment implement with an anomaly site detection function capable of performing therapy, treatment, or the like of the anomaly site while viewing an anomaly site on a tooth such as, for example, a dental caries site or a dental plaque adhesion site by using an image.

### BACKGROUND ART

Conventionally, in the field of the dental treatment, a dentist performs therapy or treatment such as the excision of a dental caries site or the removal of dental plaque and tartar by operating various dental treatment implements such as a dental air-turbine handpiece equipped with tools or the like, a micromotor handpiece, a scaler, and a three-way syringe and further determines the site concerned by using a dental detection device for detecting a dental caries site and a dental plaque adhesion site.

The foregoing dental treatment implement is required to be able to accurately detect an anomaly site on a tooth and to perform minimal intervention (MI) in the treatment of a dental caries site or the like in an efficient and reliable manner or to perform professional mechanical tooth cleaning (PMTC) in brushing guidance and treatment in an efficient and reliable mariner.

Patent Document 1 proposes a plaque detection device including a camera for photographing an oral cavity, a light source for emitting irradiation light into the oral cavity, and an image information processing means for extracting an anomaly site such as a dental plaque adhesion site on the basis of a difference in color component in image data photographed by the camera, wherein an employed light source is an LED light source that emits light in the wavelength band around 405 nm including the wavelength of 405 nm.

The plaque detection device, however, does not have a treatment function for a dental plaque adhesion site or the like, though having a detection function therefor.

### CITATION LIST

### Patent Documents

Patent Document 1: Japanese Patent Application Laid-Open No. 2011-182933

### SUMMARY OF THE INVENTION

### Problems to be Solved by the Invention

A problem to be solved by the present invention is that there is no dental treatment implement with an anomaly site detection function capable of accurately detecting an anomaly site on a tooth and gingiva (marginal part) in an oral cavity using an image, accurately treating the site on the basis of the image, and performing MI and PMTC in an efficient and reliable manner.

### Means for Solving the Problems

A dental treatment implement with an anomaly site detection function according to the present invention is most primarily characterized by including: a dental treatment implement for treating an anomaly site in an oral cavity; a color camera module group including a plurality of color camera modules, wherein each color camera module is constructed by integrating a light emission means including a light source unit for at least emitting excitation light for producing fluorescent light at an anomaly site in the oral cavity and an image pick-up means for picking up an image of the anomaly site and surrounding sites in the oral cavity as a color image, wherein the wavelengths of the excitation light emitted from the light source units are different from each other, and wherein one of the color camera modules is detachably mounted in a specific position on the dental treatment implement; and a light emission/image pick-up optical system for radiating the excitation light from the light source unit of one of the color camera modules mounted in the specific position toward the anomaly site and guiding the fluorescent light produced at the anomaly site to the image pick-up means of the color camera module.

### Advantageous Effect of the Invention

According to the present invention of claim 1, it is possible to provide a dental treatment implement with an anomaly site detection function, wherein one of the color camera modules of the color camera module group, in which the wavelengths of the excitation light emitted from the light source units of the color camera modules are different from each other, is detachably mounted in a specific position in the dental treatment implement, the dental treatment implement including a light emission/image pick-up optical system for radiating the excitation light from the light source unit of the color camera module toward the anomaly site and guiding the fluorescent light produced at the anomaly site to the image pick-up means of the color camera module, thereby enabling the function of detecting an anomaly site of a row of teeth to be implemented in various forms and enabling efficient and reliable implementation of MI in treatment of a dental caries site or the like and PMTC in brushing guidance and treatment of a row of teeth.

According to the present invention of claim 2, it is possible to provide a dental treatment implement with, an anomaly site detection function, the dental treatment implement including: a dental treatment implement for treating an anomaly site in an oral cavity; a color camera module group including: a first color camera module constructed by integrating a first light emission means including a light source unit for emitting monochromatic excitation light for producing fluorescent light at an anomaly site in the oral cavity and an image pick-up means for picking up an image of the anomaly site and surrounding sites in the oral cavity as a color image; a second color camera module constructed by integrating a second light emission means formed by combining a light source unit for emitting monochromatic excitation light for producing fluorescent light at the anomaly site in the oral cavity and a light source unit for emitting illumination light for illuminating the oral cavity in such a way as to enable changeover between the light source units or simultaneous emission thereof and an image pick-up means for picking up an image of the anomaly site and surrounding sites in the oral cavity as a color image; and a third color camera module constructed by integrating a third light emission means including a light source unit for selectively emitting excitation light of three primary colors of red, green, and blue for producing fluorescent light at the anomaly site in the oral cavity and an image pick-up means for picking up an image of the anomaly site and surrounding sites In the oral cavity as a color image, wherein the wavelengths of the excitation light emitted from the light source units of the first to third color camera modules are different from each other and wherein one of the first to third color camera modules is detachably mounted in a specific position able to be exposed to the outside or in a specific position enabling operation from the outside by disassembly of the dental treatment implement; and a light emission/image pick-up optical system for radiating the excitation light from the light source unit of one of the color camera modules mounted in the specific position toward the anomaly site and guiding the fluorescent light produced at the anomaly site to the image pick-up means of the color camera module, thereby enabling the function of detecting an anomaly site of a row of teeth to be implemented in various forms and enabling efficient and reliable implementation of MI in treatment of a dental caries site or the like and PMTC in brushing guidance and treatment of a row of teeth.

According to the present invention of claim 3, it is possible to provide a dental treatment implement with an anomaly site detection function, the dental treatment implement including: a dental treatment implement for treating an anomaly site in an oral cavity; a color camera module group including: a first color camera module constructed by integrating a first light emission means including a light source unit for emitting blue, green, or red excitation light for producing fluorescent light at an anomaly site in the oral cavity and an image pick-up means for picking up an image of the anomaly site and surrounding sites in the oral cavity as a color image; a second color camera module constructed by integrating a second light emission means formed by combining a light source unit for emitting blue, green, or red excitation light for producing fluorescent light at the anomaly site in the oral cavity and a light source unit for emitting illumination light for illuminating the oral cavity in such a way as to enable changeover between the light source units or simultaneous emission thereof and an image pick-up means for picking up an image of the anomaly site and surrounding sites in the oral cavity as a color image; and a third color camera module constructed by integrating a third light emission means including a light source unit for selectively emitting excitation light of three primary colors of red, green, and blue for producing fluorescent light at the anomaly site in the oral cavity and an image pick-up means for picking up an image of the anomaly site and surrounding sites in the oral cavity as a color image, wherein one of the first to third color camera modules is detachably mounted in a specific position able to be exposed to the outside or in a specific position enabling operation from the outside by disassembly of the dental treatment implement; and a light emission/image pick-up optical system for radiating the excitation light from the light source unit of one of the color camera modules mounted in the specific position toward the anomaly site and guiding the fluorescent light produced at the anomaly site to the image pick-up means of the color camera module, thereby enabling the function of detecting an anomaly site of a row of teeth to be implemented in various forms and enabling efficient and reliable implementation of MI in treatment of a dental caries site or the like and PMTC in brushing guidance and treatment of a row of teeth.

According to the present invention of claim 4, it is possible to provide a dental treatment implement with an anomaly site detection function, wherein an angle-type or straight-type micromotor handpiece or an angle-type or straight-type air-turbine handpiece is employed as the dental treatment implement, wherein each color camera module is detachably arranged in a position able to be exposed to the outside by disassembly in the inside of the handpiece body, and wherein the light emission/image pick-up optical system is arranged between the position near the head part where the cutting tool in the handpiece body is arranged and the color camera module, thereby enabling the function of detecting an anomaly site of a row of teeth to be implemented in various forms and enabling efficient and reliable implementation of MI in treatment of a dental caries site or the like and PMTC in brushing guidance and treatment of a row of teeth.

According to the present invention of claim 5, it is possible to provide a dental treatment implement with an anomaly site detection, function, wherein an angle-type or straight-type micromotor handpiece or an angle-type or straight-type air-turbine handpiece is employed as the dental treatment implement and wherein the color camera module and the light emission/image pick-up optical system are integrated and are formed as one of three types of color camera module units each of which is detachably in the position near the head part where a cutting tool in the handpiece body is arranged, thereby enabling the function of detecting an anomaly site of a row of teeth to be implemented in various forms and enabling efficient and reliable implementation of MI in treatment of a dental caries site or the like and PMTC in brushing guidance and treatment of a row of teeth.

According to the present invention of claim 6, it is possible to provide a dental treatment implement with an anomaly site detection function, wherein the color camera module is detachably provided with a filter for cutting the wavelength components of the excitation light among lights incident onto the image pick-up means, thereby enabling a clearer color image to be obtained with the excitation light components cut out.

### BRIEF DESCRIPTION OF THE DRAWINGS

FIG. 1 is a partially cutaway front view of a micromotor handpiece with an anomaly site detection function according to a first embodiment of the present invention.

FIG. 2 is a top plan view of the micromotor handpiece with the anomaly site detection function according to the first embodiment.

FIG. 3 is a schematic block diagram of a color camera module in the micromotor handpiece with the anomaly site detection function according to the first embodiment.

FIG. 4 is a schematic end view of the color camera module in the micromotor handpiece with the anomaly site detection function according to the first embodiment.

FIG. 5 is a schematic block diagram of a camera unit in the micromotor handpiece with the anomaly site detection function according to the first embodiment.

FIG. 6 is a sectional view taken along line A-A of FIG. 5.

FIG. 7 is an enlarged explanatory diagram of a rod fiber in the micromotor handpiece with the anomaly site detection function according to the first embodiment.

FIG. 8 is an explanatory diagram of an optical arrangement of the camera unit, the rod fiber, and an objective lens in the micromotor handpiece with the anomaly site detection function according to the first embodiment.

FIG. 9 is an explanatory diagram illustrating a detachable structure of first to third color camera modules from a coupling part in the micromotor handpiece with the anomaly site detection function according to the first embodiment.

FIG. 10 is a schematic block diagram of the first to third color camera modules in the micromotor handpiece with the anomaly site detection function according to the first embodiment.

FIG. 11 is a schematic block diagram of a filter used in the micromotor handpiece with the anomaly site detection function according to the first embodiment.

FIG. 12 is a circuit configuration diagram illustrating a driving circuit system of a light source unit of the first color camera module in the micromotor handpiece with the anomaly site detection function according to the first embodiment.

FIG. 13 is a circuit configuration diagram illustrating a driving circuit system of a light source unit of the second color camera module in the micromotor handpiece with the anomaly site detection function according to the first embodiment.

FIG. 14 is a circuit configuration diagram illustrating a driving circuit system of a light source unit of the third color camera module in the micromotor handpiece with the anomaly site detection function according to the first embodiment.

FIG. 15 is a block diagram illustrating a control unit including a light emitting element drive system and an image processing system related to the micromotor handpiece with the anomaly site detection function according to the first embodiment.

FIG. 16 is a graph schematically illustrating a relationship between the wavelength and the intensity of excitation light or fluorescent light at the time of detection of a dental caries site in the micromotor handpiece with the anomaly site detection function according to the first embodiment.

FIG. 17 is an explanatory diagram schematically illustrating a state of an affected area where fluorescent light is produced wherein the fluorescent light corresponds to excitation light at the time of detection of the dental caries site in the micromotor handpiece with the anomaly site detection function according to the first embodiment.

FIG. 18 is an explanatory diagram schematically illustrating a state of an affected area where fluorescent light is produced wherein the fluorescent light corresponds to excitation light at the time of detection of the dental plaque adhesion site in the micromotor handpiece with the anomaly site detection function according to the first embodiment.

FIG. 19 is a partially cutaway front view of a micromotor handpiece with an anomaly site detection function according to a variation of the first embodiment of the present invention.

FIG. 20 is a partially cutaway front view of an air-turbine handpiece with an anomaly site detection function according to a second embodiment of the present invention.

FIG. 21 is an explanatory diagram illustrating a detachable structure of first to third color camera modules from a coupling part in the air-turbine handpiece with the anomaly site detection function according to the second embodiment.

### MODE FOR CARRYING OUT THE INVENTION

The present invention achieves an object to provide a dental treatment implement with an anomaly site detection function capable of accurately detecting an anomaly site on a tooth and gingiva (marginal part) in an oral cavity using an image, accurately treating the site on the basis of the image, and performing MI and PMTC in an efficient and reliable manner by using a configuration having: a color camera module group including: a first color camera module constructed by integrating a first light emission means including a light source unit for emitting monochromatic excitation light for producing fluorescent light at an anomaly site in the oral cavity and an image pick-up means for picking up an image of the anomaly site and surrounding sites in the oral cavity as a color image; a second color camera module constructed by integrating a second light emission means formed by combining a light source unit for emitting monochromatic excitation light for producing fluorescent light at the anomaly site in the oral cavity and a light source unit for emitting illumination light for illuminating the oral cavity in such a way as to enable changeover between the light source units or simultaneous emission thereof and an image pick-up means for picking up an image of the anomaly site and surrounding sites in the oral cavity as a color image; and a third color camera module constructed, by integrating a third light emission means including a light source unit for selectively emitting excitation light of three primary colors of red, green, and blue for producing fluorescent light at the anomaly site in the oral cavity and an image pick-up means for picking up an image of the anomaly site and surrounding sites in the oral cavity as a color image, wherein the wavelengths of the excitation light emitted, from the light source units of the first to third color camera modules are different from each other and wherein one of the first to third color camera modules is detachably mounted in a specific position able to be exposed to the outside or in a specific position enabling operation from the outside by disassembly of the dental treatment implement; and a light emission/image pick-up optical system for radiating the excitation light from the light source unit of one of the color camera modules mounted in the specific position toward the anomaly site and guiding the fluorescent light produced at the anomaly site to the image pick-up means of the color camera module.

### EMBODIMENTS

A dental treatment implement with an anomaly site detection function according to an embodiment of the present invention will be described in detail hereinafter with reference to accompanying drawings.

### First Embodiment

A dental treatment implement with, an anomaly site detection function according to a first embodiment is applied to a micromotor-driven-type handpiece which is a kind of dental treatment instrument.

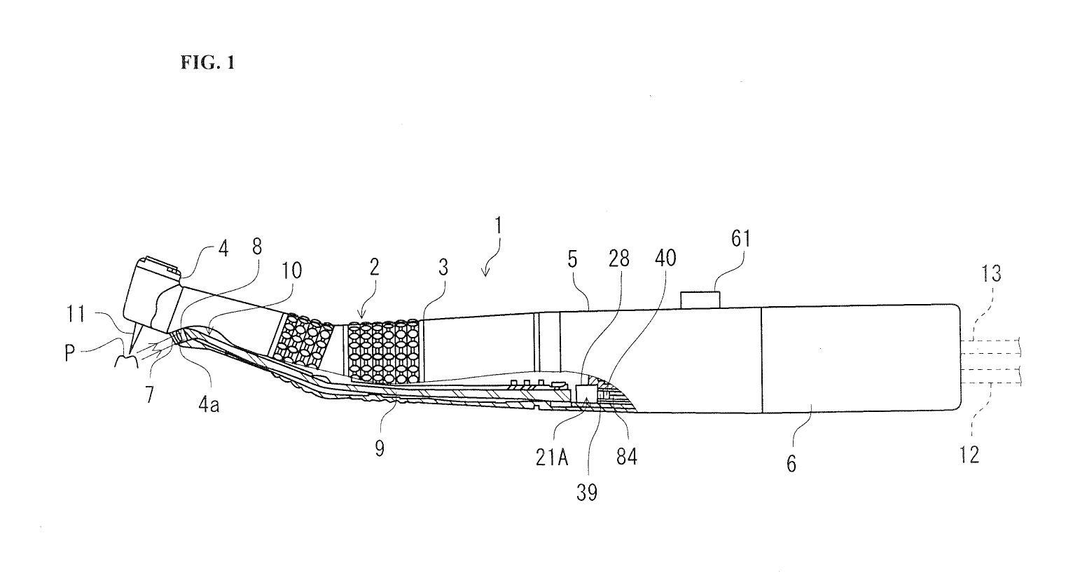

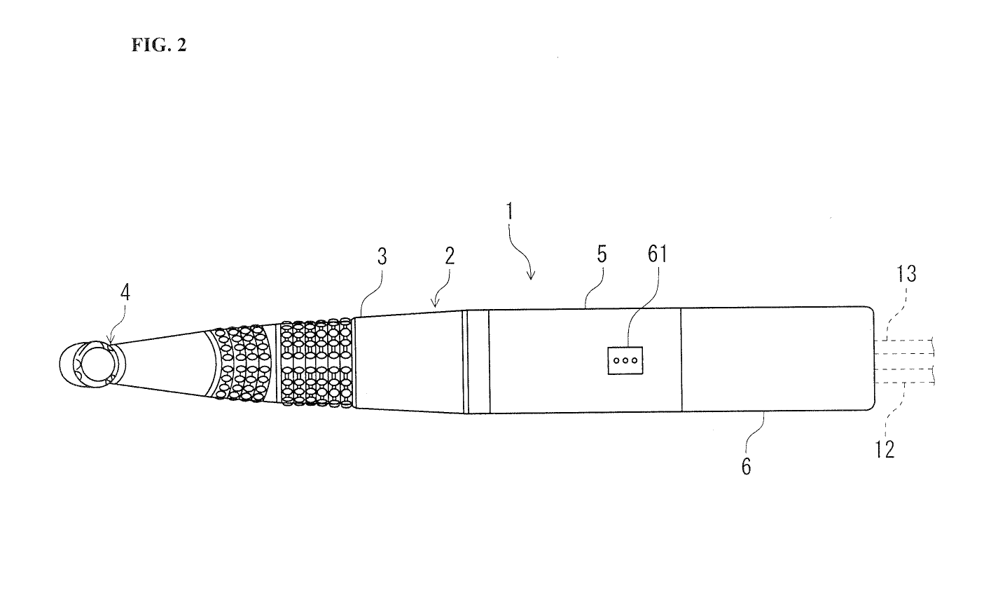

As illustrated in FIGS. 1 and 2, the micromotor handpiece 1 with the anomaly site detection function according to the first embodiment (hereinafter, referred to as “handpiece 1 of the first embodiment”) includes: a handpiece body 2 which includes a grip part 3 having a head part 4, where a cutting tool 11 for treating an affected area P is detachably mounted, on the distal end side and a coupling part 5 which is formed in a substantially cylindrical shape and detachably coupled to the grip part 3 and which detachably houses whole of one of color camera modules (for example, a first color camera module 21A) of a color camera module group 21 composed of three kinds of color camera modules, i.e., first to third color camera modules 21A, 21B, and 21C, having detachable compatibility and described later in detail, at the inside bottom by attachment or detachment to or from, for example, a holder 28 provided in the coupling part 5 and by attachment or detachment between a mounting connector 39 provided on the first color camera module 21A side and a receiving connector 40 provided on the coupling part 5 side; and a micromotor 6 for rotationally driving the cutting tool 11 detachably mounted at the rear end of the coupling part 5 in the handpiece body 2 at high speed.

In the inside of the grip part 3, there is arranged a light emission/image pick-up optical system 10 including: an image pick-up window 7, which is formed of, for example, transparent glass material or transparent synthetic resin material, arranged in the position near the head, part 4 and toward the cutting site of the cutting tool 11; an objective lens 8 arranged in the inside of the image pick-up window 7; and a rod fiber 3 which is a light, guide member with a light incident end facing the near area of the objective lens 8 and the light emitting end facing the color camera module 21,

The handpiece 1 of the first embodiment is further connected to a signal output cable 12 and a light emitting element driving cable 13, which will be described in detail later.

The connection locations of the signal output cable 12 and the light emitting element driving cable 13 to the handpiece 1 of the first embodiment are not particularly limited. It is, however, possible to give an example such that connectors are provided in appropriate locations of the coupling part 5 for connection to the handpiece 1.

Detailed description will, be omitted here for an electric and mechanism drive system of the cutting tool 11 to be mounted on the head part 4.

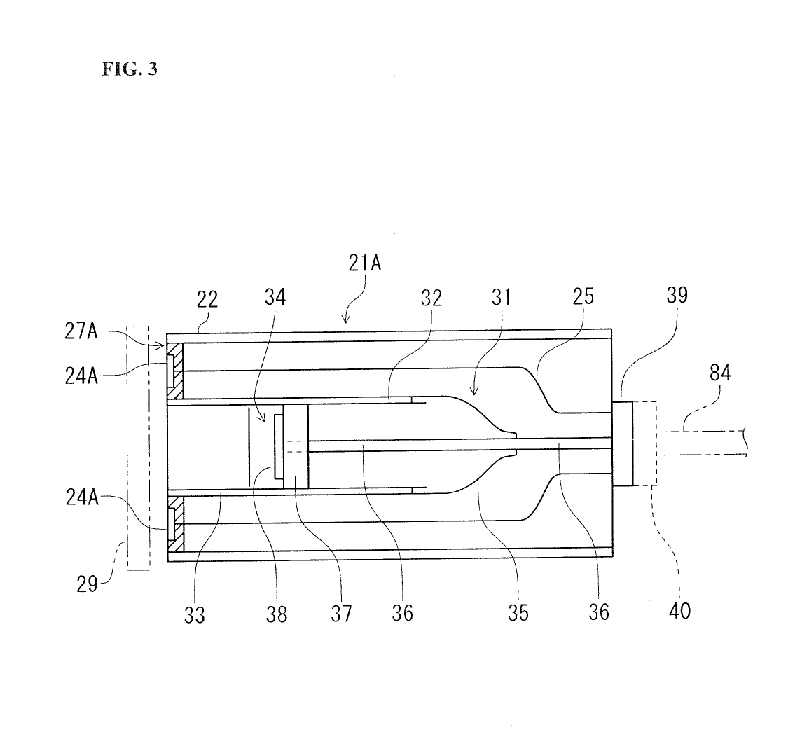

Subsequently, the first to third color camera modules 21A, 21B, and 21C will be described with reference to FIGS. 3 to 14.

First, the first color camera module 21A will be described.

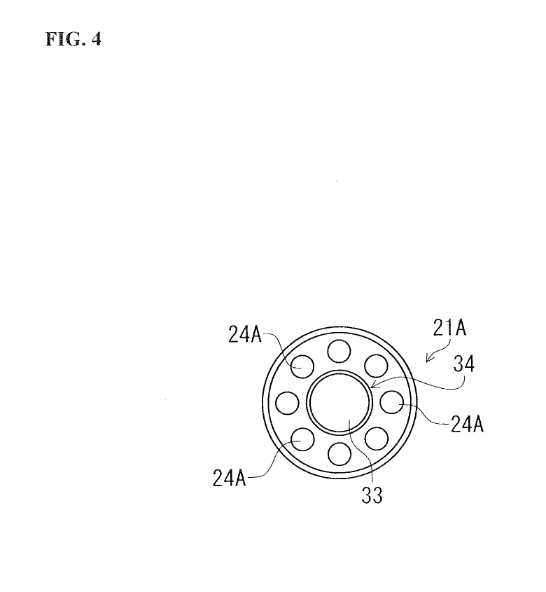

The first color camera module 21A includes a cylindrical camera head part 22 as illustrated in FIGS. 3 and 4 and the camera head part 22 houses a camera unit 31 which is arranged inwardly from the distal end surface and a light source unit 27A constituting a light emission, means where an arbitrary number of (for example, eight) light emitting elements (LED: light emitting diodes) 24A (such as those of driving voltage DC 3.3V) of the same kind are arranged in a circle and integrated around the end surface of the camera unit 31, where the light emitting elements 24A emit monochromatic excitation light (the excitation light in the blue region of the wavelength 470±30 nm).

As the light emitting elements 24A, it is also possible to use LEDs emitting excitation light in the green region or LEDs emitting excitation light in the red region.

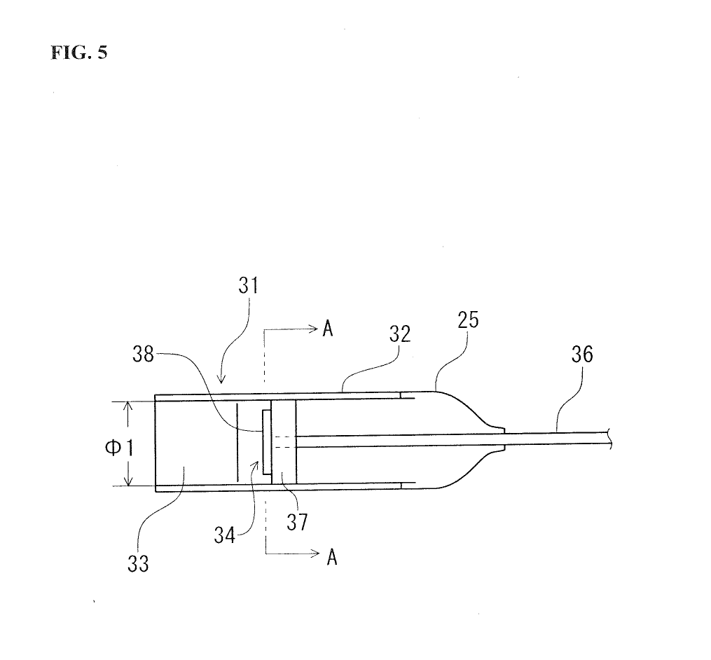

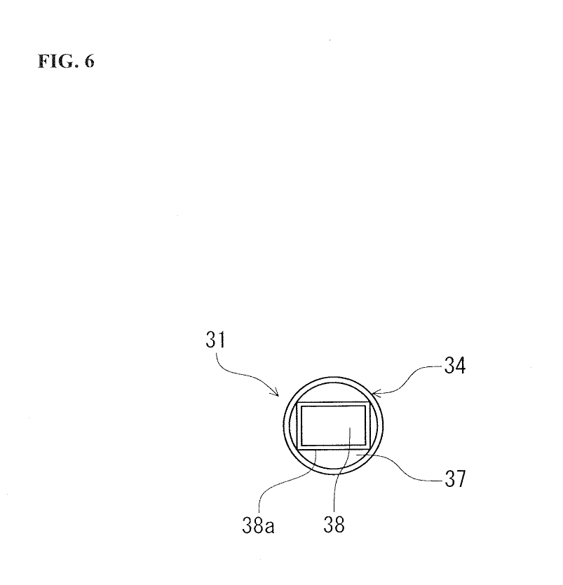

The camera unit 31 will be described in detail below with reference to FIGS. 5 and 6.

The camera unit 31 includes: a cylindrical support cylinder 32, for example, having a diameter of 1.2 mm, an inner diameter of about 1.1 mm, and a length of 3 mm; a condenser lens unit 33 having a diameter Φ1 of 1.1 mm with a light incident end arranged to face one end surface of the support cylinder 32; an image pick-up unit 34 oppositely arranged at a fixed, interval apart from the condenser lens unit 33 in the support cylinder 32; a cover member 35 which is fitted in a range from the other end surface side of the support cylinder 32 to the inside of the support cylinder 32; and a signal cable 36 which is connected to the image pick-up unit 34 and led to the rear side through the cover member 35.

The image pick-up unit 34 includes a disk-shaped support substrate 37 having a diameter of 1.1 mm and firmly fixed with the center aligned with the optical axis of the condenser lens unit 33 in the support cylinder 32 and a color image sensor (CMOS: complementary metal oxide semiconductor) 38 attached in a state where color pixels are arranged in a matrix of 320×240 pixels as the number of pixels on the surface of a sensor substrate 38a having external dimensions of 0.84×0.74 mm and a thickness of 0.1 mm and where the center portion coincides with the optical axis of the condenser lens unit 33, wherein the signal cable 36 is connected to the color image sensor 38 at one end and the other end thereof is led to the rear side through the support substrate 37 and the cover member 35.

The condenser lens unit 33 is, for example, one having optical characteristics of a viewing angle of 70 degrees and a focus range of 3 to 50 mm.

One end of the light emitting element cable 25 is connected to each of the light emitting elements 24A, which constitute the light source unit 27A, arranged around the end surface of the camera unit 31.

Furthermore, the light emitting element cable 25 and the signal cable 36 are connected, to a mounting connector 39. In addition, the signal output cable 12 and the light emitting element driving cable 13 are connected to the receiving connector 40 via a camera cable 84.

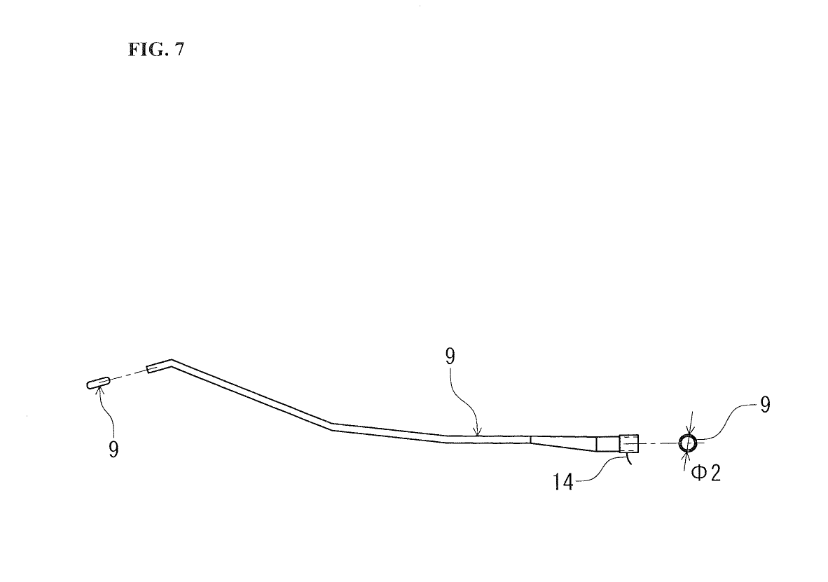

As illustrated in FIG. 7, multi-component glass is used, for all of the core, cladding, and skin tube of the rod fiber 9 and the rod fiber 9 has a step index type structure that provides different refractive indices in stages and has optical characteristics of a light receiving angle of about 70 degrees and a numerical aperture (NA) of 0.57.

In the rod fiber 9, the shape of the light emitting end thereof has, for example, a fiber diameter Φ2 of about 2.4 mm, the light incident end has an oval shape, for example, with a major axis of about 3.6 mm and a minor axis of about 1.35 mm, and the outer periphery of the end portion of the light emitting end is covered with a stainless-steel cylindrical body 14. Moreover, the rod fiber 9 has a specification with autoclave resistance of within 90% with respect to an initial transmittance after 350 cycles under the autoclave conditions of 135° C., 100% RH, and 3 minutes.

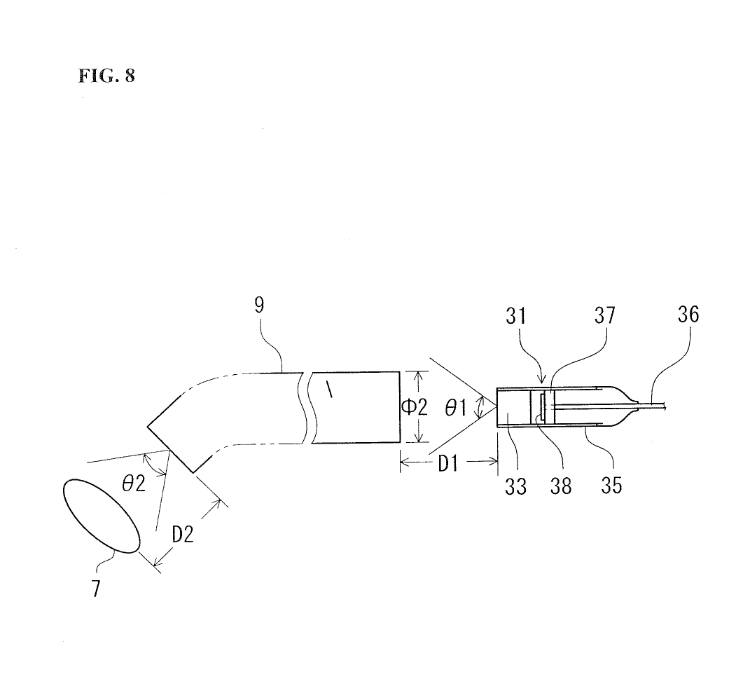

Here, the optical detailed structure of the camera, unit 31 included in the color camera module 21A, the rod fiber 9, and the objective lens 8 will be described in detail with reference to the enlarged explanatory diagram of FIG. 8.

Regarding the relationship between the light emitting end of the rod fiber 9 and the condenser lens unit 33 of the camera unit 31, a condenser lexis unit having a viewing angle θ1 of 70 degrees is used as the condenser lens unit 33, and an interval D1 between the light incident surface of the condenser lens unit 33 and the light emitting end of the rod fiber 9 is set to about 3 mm by employing the light emitting end of the rod fiber 9 having a diameter Φ2 of about 2.4 mm, thereby enabling a beam emitted from the light emitting end of the rod fiber 9 to be received within the range of the viewing angle of the condenser lens unit 33 and thus enabling the light to be guided to the condenser lens unit 33 without hindrance.

On the other hand, regarding the relationship between the objective lens 8 and the light incident end of the rod fiber 9, the light receiving angle θ2 of the rod fiber 42 is about 70 degrees and therefore, for example, a convex lens having a lens diameter on the order of 3 mm and a focal length on the order of 3 mm is used as the objective lens 8 and the interval D2 between the objective lens 3 and the light incident end of the rod fiber 9 is set to about 3 mm, thereby enabling the image pick-up light incident onto the light incident end of the rod fiber 9 via the objective lens 8 to be received, within the range of the light receiving angle and thus enabling the light to be guided to the light, incident end of the rod fiber 9 without hindrance.

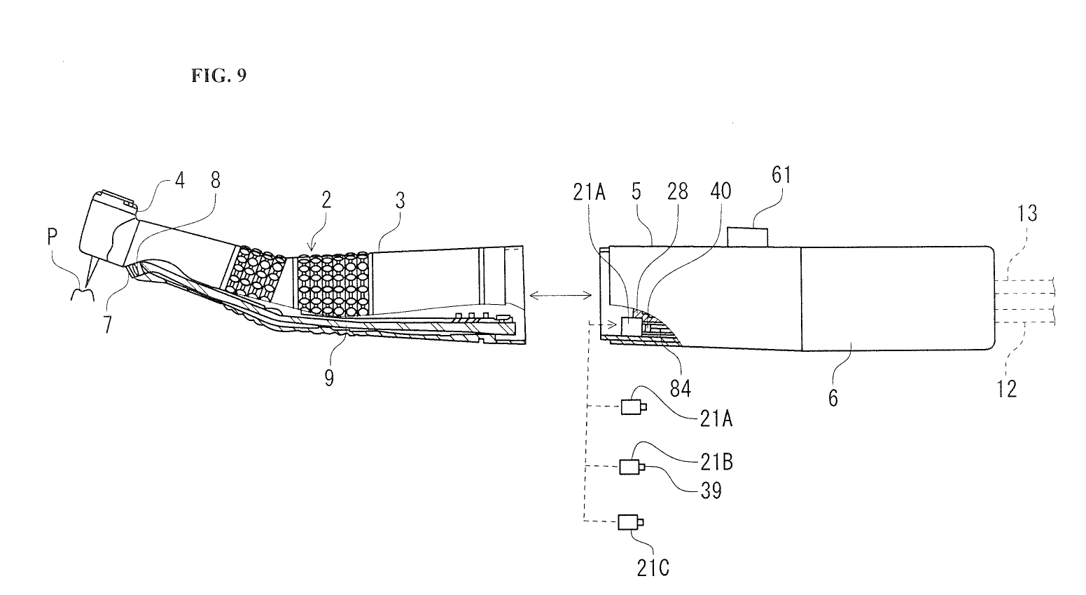

Subsequently, the detachable structure of the first to third color camera modules 21A, 21B, and 21C from the coupling part 5 will be described in further detail with reference to FIG. 9.

As illustrated in FIG. 9, the configuration of the first color camera module 21A among the first to third color camera modules 21A, 21B, and 21C is as described above. Moreover, each of the second color camera module 21B and the third color camera module 21 is also configured in the same manner as the first color camera module 21A.

Then, as illustrated in FIG. 9, any one of the first to third color camera modules 21A, 21B, and 21C is selectively and detachably arranged in the inside bottom of the coupling part 5.

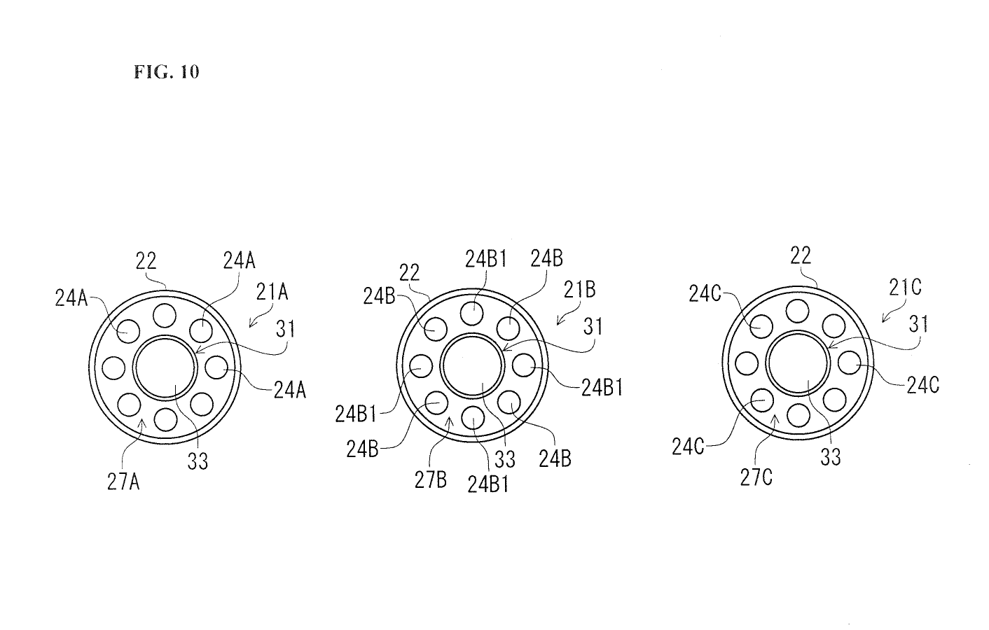

The following describes the light source units 27A to 27C of the first to third color camera modules 21A, 21B, and 21C with reference to FIG. 10.

The configuration of the light source unit 27A of the first color camera module 21A is as has been described.

The second color camera module 21B includes a camera unit 31 similar to that of the first color camera module 21A and a light source unit 27B constituting a light emission means where an arbitrary number of (for example, four) light emitting elements 24B of the same kind and an arbitrary number of (for example, four) light emitting elements 24B1 are arranged in a circle and integrated around the end surface of the camera unit 31, where the light emitting elements 24B emit monochromatic excitation light (the excitation light in the blue region of the wavelength 470±30 nm) and the light emitting elements 24B1 emit illumination light (illumination light of daylight color) for illuminating the oral cavity.

As the light, emitting elements 24B, it is also possible to use LEDs emitting excitation light in the green region or LEDs emitting excitation light in the red region.

The third color camera module 210 includes a camera unit 31 similar to that of the first color camera module 21A and a light source unit 27C constituting a light emission means where an arbitrary number of (for example, eight) three-primary-color light emitting elements 24C are arranged in a circle and integrated around the end surface of the camera unit 31, where the light emitting elements 24C emit three-primary-color excitation light.

The three-primary-color light emitting element 24C integrates light emitting element chips 24B1, 24B2, and 24B3 which emit excitation light in the blue region of 470±30 nm, in the green region of 530±40 nm, and in the red region, of 700±100 nm to selectively emit excitation light in each of the colors.

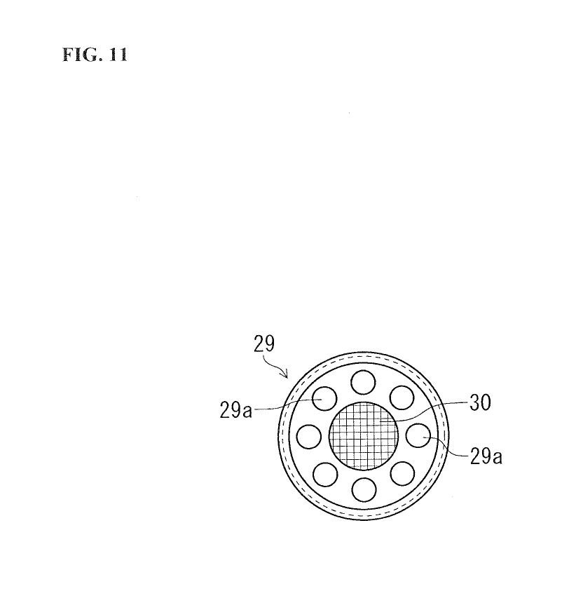

FIG. 11 illustrates a filter 29 to be mounted on, for example, the end surface of the first color camera module 21A.

The filter 29 is disk-shaped and attachable/detachable to/from the end surface of the first color camera module 21A and includes eight through-holes 29a arranged, in a circle corresponding to the arrangement of the light emitting elements 24A and a filter part 30 having light-shielding characteristics (for example, transmitting light of a wavelength of 520 nm or longer) enabling the cutting of the wavelength components of the monochromatic excitation light (the excitation light in the blue region of a wavelength of 470±30 nm) in the central portion,

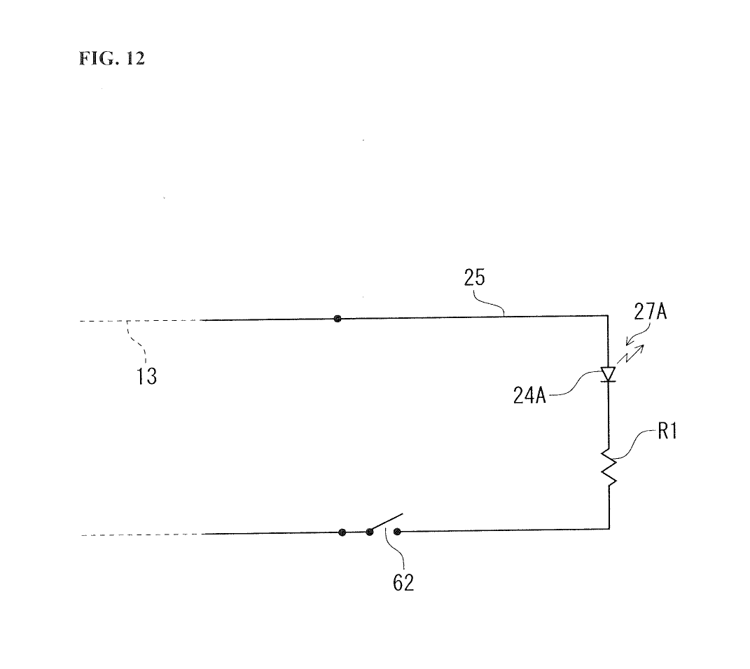

The following describes the driving circuit system of the light source units 27A to 27C in the first to third color camera modules 21A, 21B, and 21C with reference to FIGS. 12 to 14.

FIG. 12 illustrates the driving circuit system of the light source unit 27A of the first color camera module 21A, giving an example of driving one light emitting element 24A to illuminate.

In this driving circuit system, a switch 62 provided in a lighting operation unit 61 is connected between the light emitting element driving cable 13 (two-wire configuration) and the light emitting element cable 25 (two-wire configuration) and the light emitting element cable 25 is connected to a series circuit including the light emitting element 24 and the current-limiter resistor R1, by which the on operation of the switch 62 causes a predetermined, voltage to be applied between an anode and a cathode of the light, emitting element 24 to turn on the light emitting element 24A so as to emit excitation light and the off-operation of the switch 62 turns off the light emitting element 24.

In practice, a lighting driving circuit system is manufactured and used with respect to eight light emitting elements 24A on the basis of the configuration similar to the configuration illustrated in FIG. 12.

Although the lighting operation unit 61 can be mounted on, for example, the outer surface of the coupling part 5 as illustrated in FIG. 1, the arrangement is not particularly limited thereto.

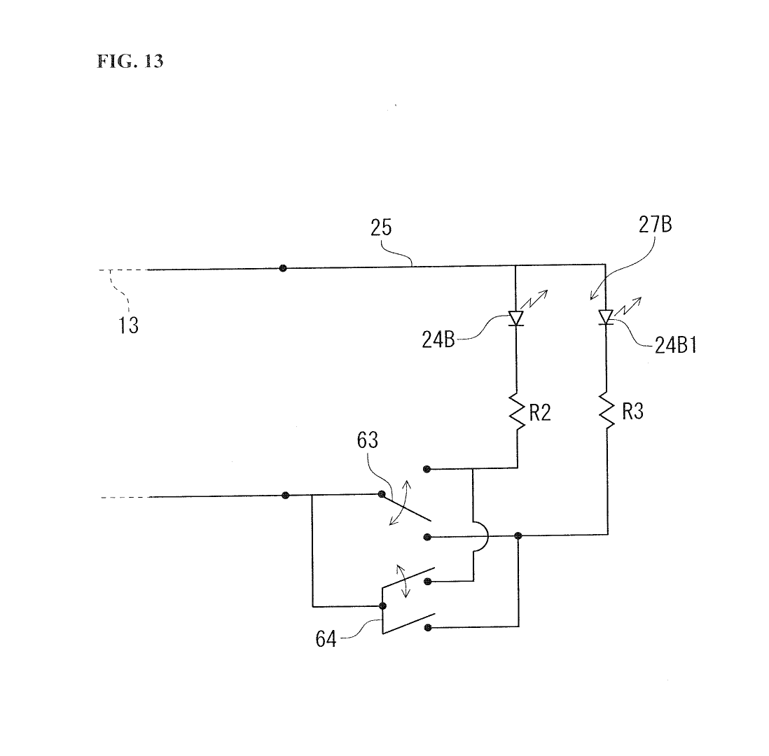

FIG. 13 illustrates a driving circuit system for the light source unit 2B in the second color camera module 21B by giving an example of driving one light emitting element 24B and one light emitting element 24B1 to illuminate.

In this driving circuit system, a changeover switch 63 for switching between two circuits and a full lighting switch 64 provided in the lighting operation unit 61 are connected between the light emitting element driving cable 13 and the light emitting element cable 25 and the changeover switch 63 is used for switching between a series circuit including the light emitting element 24B and the current-limiter resistor R2 and a series circuit including the light emitting element 24B1 and the current-limiter resistor R3, thereby causing a predetermined voltage to be selectively applied between an anode and a cathode of the light emitting element 24B or between an anode and a cathode of the light emitting element 24B1 to emit excitation light from the light emitting element 24B or to emit illumination light from the light emitting element 24B1.

Moreover, the full lighting switch 64 brings lead terminals from both of the series circuits into conduction simultaneously to the light emitting element driving cable 13 in order to perform the light emission, from the light emitting element 24B and the light emission from the light emitting element 24B1 at the same time.

In practice, a lighting driving circuit system is manufactured and used with respect to four light emitting elements 24B and four light emitting elements 24B1 on the basis of the configuration similar to the configuration illustrated in FIG. 13.

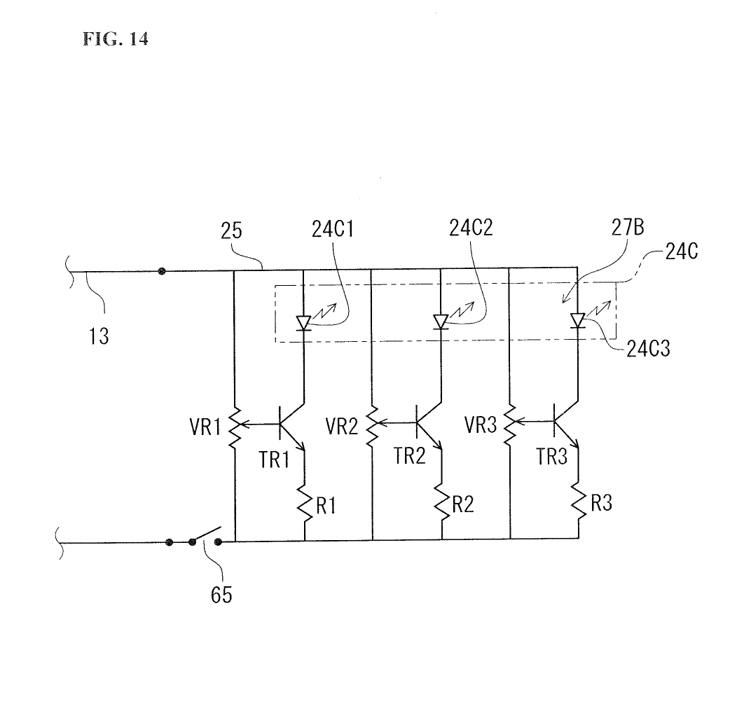

FIG. 14 illustrates a driving circuit system for the light source unit 27C in the third, color camera module 21C by giving an example of driving one three-primary color light emitting element 24C to illuminate.

The three-primary-color light emitting element 24C includes a light emitting element chip 24C1 for emitting excitation light in the blue region of 470±30 nm; a light emitting element chip 24C2 for emitting excitation light in the green region of 530±40 nm, and a light emitting element chip 24C3 for emitting excitation light in the red region of 700±100 nm wavelength.

This driving circuit system is provided with a switch 65 between the light emitting element driving cable 13 and the light emitting element cable 25.

Additionally, the light emitting element cable 25 is connected to a series circuit including the light emitting element chip 24C1, a lighting control transistor TR1, and a current-limiter resistor R4, a series circuit including the light emitting element chip 24C2, a lighting control transistor TR2, and a current-limiter resistor R5, and a series circuit including the light emitting element chip 24C3, a lighting control transistor TR3, and a current-limiter resistor R6.

Furthermore, three current adjusting variable resistors VR1, VR2, and VR2 connected to the light emitting element cable 25 adjust the respective base currents of the lighting control transistors TR1, TR2, and TR3, thereby enabling adjustment of the light emission amounts of the excitation light in the blue region, the excitation light in the green region, and the excitation light in the red region.

In practice, a lighting driving circuit system is manufactured and used with respect to eight three-primary-color light emitting elements 24C on the basis of the configuration similar to the configuration illustrated in FIG. 14.

In addition, the lighting operation unit 61 has been described separately in the above description. In practice. however, the lighting operation unit 61 is configured so as to be compatible with the foregoing three aspects and mounted, for example, on the outer surface of the coupling part 5.

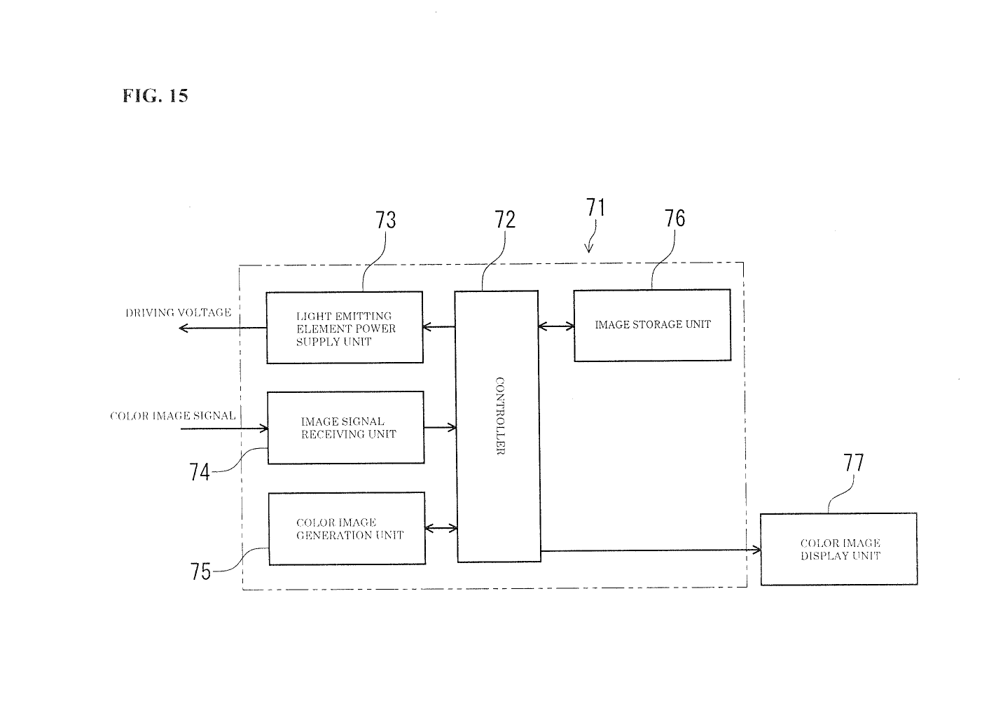

FIG. 15 illustrates a control unit 71 including a light emitting element drive system and an image processing system, related to the handpiece 1 of the first embodiment.

The control unit 71 includes a controller 72 which controls the entire operation, a light emitting element power supply unit 73 which supplies a driving voltage to each of the light emitting elements, an image signal, receiving unit 74 which receives an image signal, from the camera unit 31 arranged in the handpiece 1, a color image generation unit 75 which generates a color image of a tooth and gingiva (marginal part) in the oral cavity on the basis of the received image signal, and an image storage unit 76 which stores the generated color image.

Furthermore, the control unit 71 has a color image display unit 77 including a color liquid crystal display or the like which displays a color image picked up by the camera unit 31 and generated by the color image generation unit 75 on the screen.

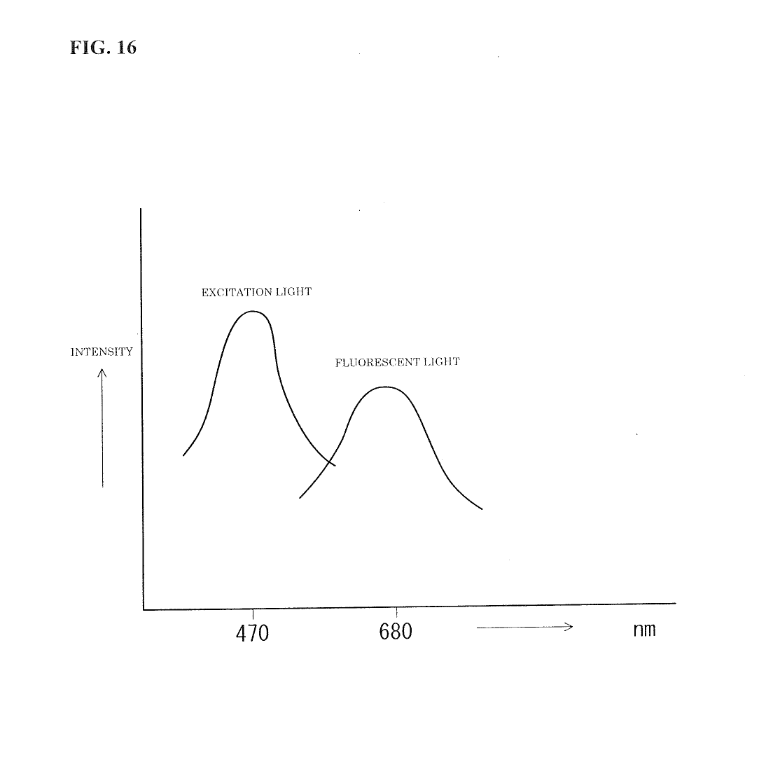

Subsequently, the anomaly site detection function for a row of teeth implemented by the handpiece 1 of the first embodiment will be described with reference to FIGS. 16, 17, and 18.

For example, if the switch 62 is turned on in a state where the first color camera module 21A is mounted on the coupling part 5 in the handpiece body 2 as illustrated in FIG. 1 in the handpiece 1 of the first embodiment and the head part 4 faces the oral cavity of a patient, the light emitting element 24A of the light source unit 27A is turned on, thereby causing the excitation light (having a wavelength of 470±30 nm) in the blue region emitted from the light emitting element 24A to be emitted, to the row of teeth in the oral cavity through the rod fiber 9, the objective lens 8, and the image pick-up window 7.

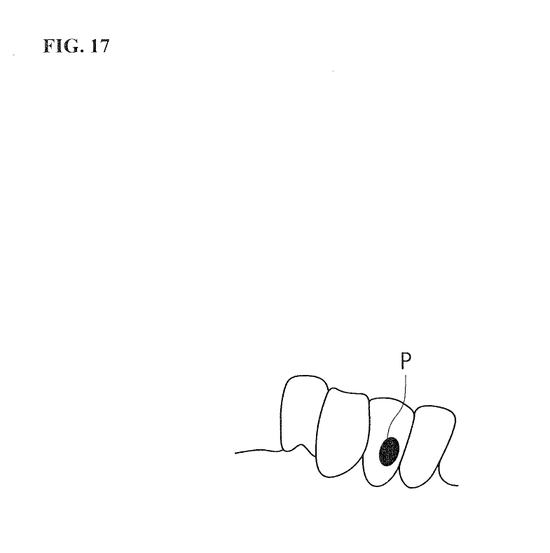

Thereby, when a dental caries site (affected area P) is present in a particular tooth in she row of teeth, fluorescent light (indicated by black in FIG. 17: for example, fluorescent light having a wavelength of 620 nm) corresponding to the excitation light is produced in the affected area P.

The wavelengths of the excitation light and fluorescent light and the intensities of the excitation light and fluorescent light in the above case are schematically illustrated in FIG. 16.

Light from the region composed of the tooth and the surrounding sites thereof including the produced fluorescent light passes through she image pick-up window 7, the objective lens 8, and the rod fiber 9, further passes through the condenser lens unit 33 of the camera unit 31 of the first color camera module 21A, and reaches the image pick-up unit 34 for picking up an image.

The image pick-up signal from the image pick-up unit 34 is transmitted to the control unit 71 via the signal cable 36 and the signal output cable 12.

In the control unit 71, the color image generation unit 75 generates a color image of the tooth, and gingiva (marginal part) corresponding to the received image pick-up signal and the color image display unit 77 displays an image on the screen as a color image corresponding to FIG. 16.

In the handpiece 1 of the first embodiment described above, the excitation light in the blue region is emitted to the row of teeth in the same manner as in the above in a state where the filter 29 is mounted on the end surface of the first color camera module 21A, the image pick-up unit 34 of the camera unit 31 picks up an image of the region, composed of the tooth and the surrounding sites thereof including the produced fluorescent light. Furthermore, when the color image display unit 77 displays the image as a color image on the screen, a color image with the excitation light components in the blue region cut out by the filter 29 is able to be displayed, by which a clearer color image can be obtained.

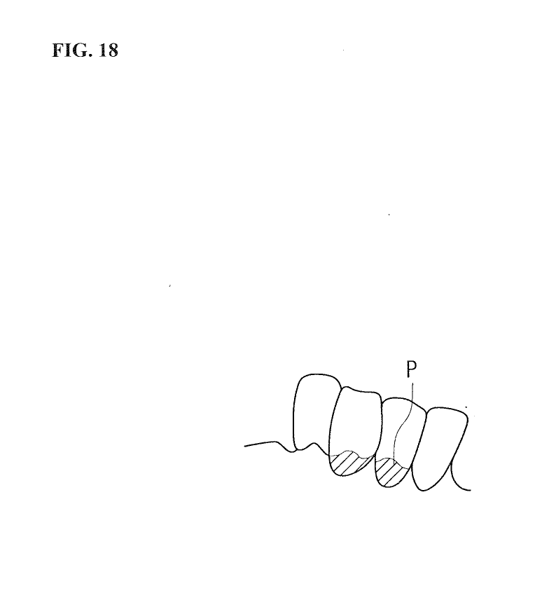

FIG. 18 illustrates an example of a dental plaque adhesion site (indicated by shaded areas) in the row of teeth whose image is picked up and displayed on the screen as a color image by the color image display unit 77 in the same manner as in the above.

The handpiece 1 of the first embodiment provides the following advantageous effects when the second color camera module 21B is mounted on the coupling part 5 in the handpiece body 2 and the same anomaly site detection operation for the row of teeth is performed in the same manner as in the above.

Specifically, when the anomaly site detection operation for the row of teeth is performed, by using only light emitted from the light emitting element 24B of the second color camera module 21B, the same advantageous effects as the above can be achieved. Moreover, the row of teeth is irradiated with the illumination light emitted only from the light emitting element 24B1, thereby enabling the second color camera module 21B to be used for illumination and image pick-up of the row of teeth.

Furthermore, the simultaneous lighting of the light emitting element 24B and the light emitting element 24B1 enables more reliable execution of the anomaly site detection operation for the row of teeth while securing sufficient brightness in the region of the row of teeth.

The handpiece 1 of the first embodiment provides the following advantageous effects when the third color camera module 21C is mounted on the coupling part 5 in the handpiece body 2 and the same anomaly site detection operation for the row of teeth, is performed in the same manner as in the above.

Specifically, the excitation light from the three-primary-color light emitting element 24C is appropriately switched between the blue region, the green region, and the red region to be emitted toward the row of teeth, thereby enabling a wide range of the anomaly site detection function to be implemented according to the kind of the affected area P or the aim of treatment.

According to the handpiece 1 of the first embodiment described in detail hereinabove, the function of detecting an anomaly site of a row of teeth can be implemented in various forms and minimal intervention (MI) can be efficiently and reliably implemented in treatment of a dental caries site or the like.

Furthermore, in the brushing guidance and treatment of the row of teeth of a patient, professional mechanical tooth cleaning (PMTC) can be efficiently and reliably performed.

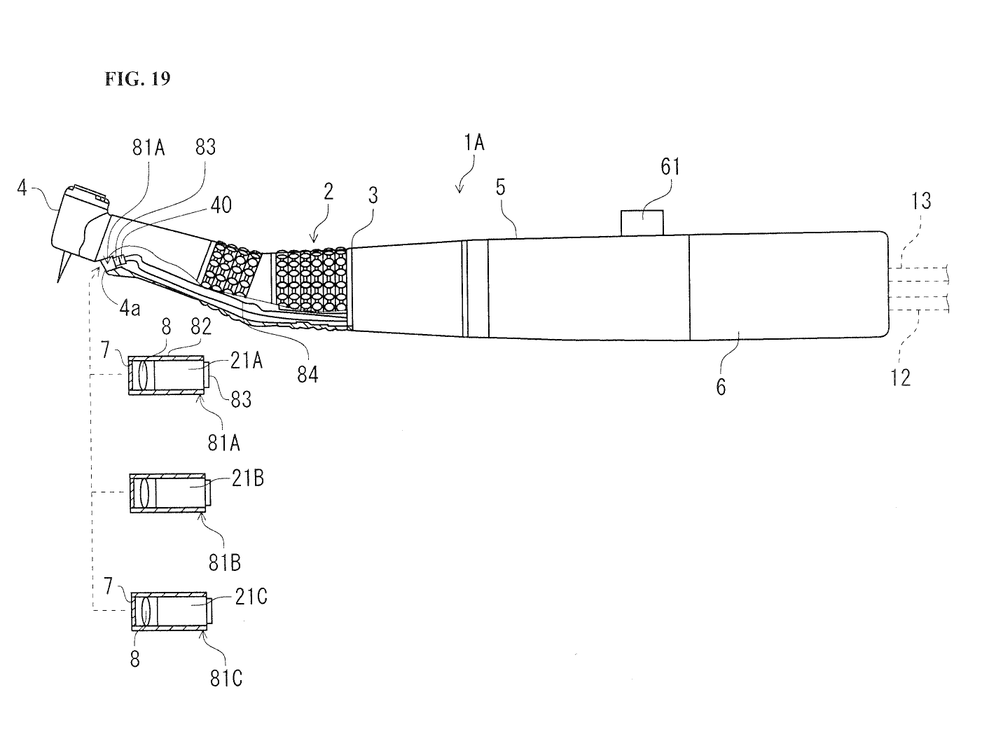

Subsequently, a handpiece 1A as a variation of the handpiece 1 of the first embodiment will be described with reference to FIG. 19.

In the handpiece 1A of the variation, the same elements as in the handpiece 1 already described are denoted by the same reference numerals, and detailed description thereof will be omitted.

Although the handpiece 1A illustrated in FIG. 19 has substantially the same configuration as that of the handpiece 1 described above, it is characterized by that first to third color camera module units 81A, 81B, and 81C are detachably arranged, on a selective basis in an opening 4a which is provided towards the cutting tool 11 side in the position near the head part 4 of the grip part 3.

Specifically, the handpiece 1A is configured in such a way that the rod fiber 9 included in the already-described, handpiece 1 is eliminated and one of a first color camera module unit 81A, a second color camera module unit 81B, and a third color camera module unit 81C is selectively and detachably arranged in the opening 4a located in the position near the head part 4, where the first color camera module unit 81A includes a first color camera module 21A and further an objective lens 8 and an image pick-up window 7 constituting a light emission/image pick-up optical system, all of which are housed in a cylinder-like body 82 so as to form an integral, unit structure, the second color camera module unit 81B includes a second, color camera module 21B, an objective lens 8, and an image pick-up window 7, all of which are housed in a cylinder-like body 82 so as to form an integral unit structure, and the third color camera module unit 81C includes a third color camera module 21C, an objective lens 8 and an image pick-up window 7, all of which are housed in a cylinder-like body 82 so as to form an integral unit structure.

Moreover, each of the first to third color camera module units 81A, 81B, and 81C includes a mounting connector 83 on the rear end side, so that the mounting connector 83 is coupled, to a receiving connector 40, which is arranged inside the grip part 3, the receiving connector 40 is connected, to one end of a camera cable 84 housing a signal cable and a light emitting element cable, and the camera cable 84 is extended to the inside of the handpiece 1A so as to be connected to the lighting operation unit 61, the signal output cable 12, and the light emitting element driving cable 13 in the same manner as in the above.

According to the handpiece 1A as the variation illustrated in FIG. 19, the first to third color camera module units 81A, 81B, and 81C are selectively mounted in the opening 4a provided in the position near the head part 4 to perform various anomaly site detection operations for a row of teeth similar to those described above, thereby also enabling the same advantageous effects to be provided. Moreover, similarly to the above, the handpiece 1A also enables efficient implementation of MI in treatment of a dental caries site or the like and efficient implementation of PMTC in brushing guidance and treatment of a row of teeth.

### Second Embodiment

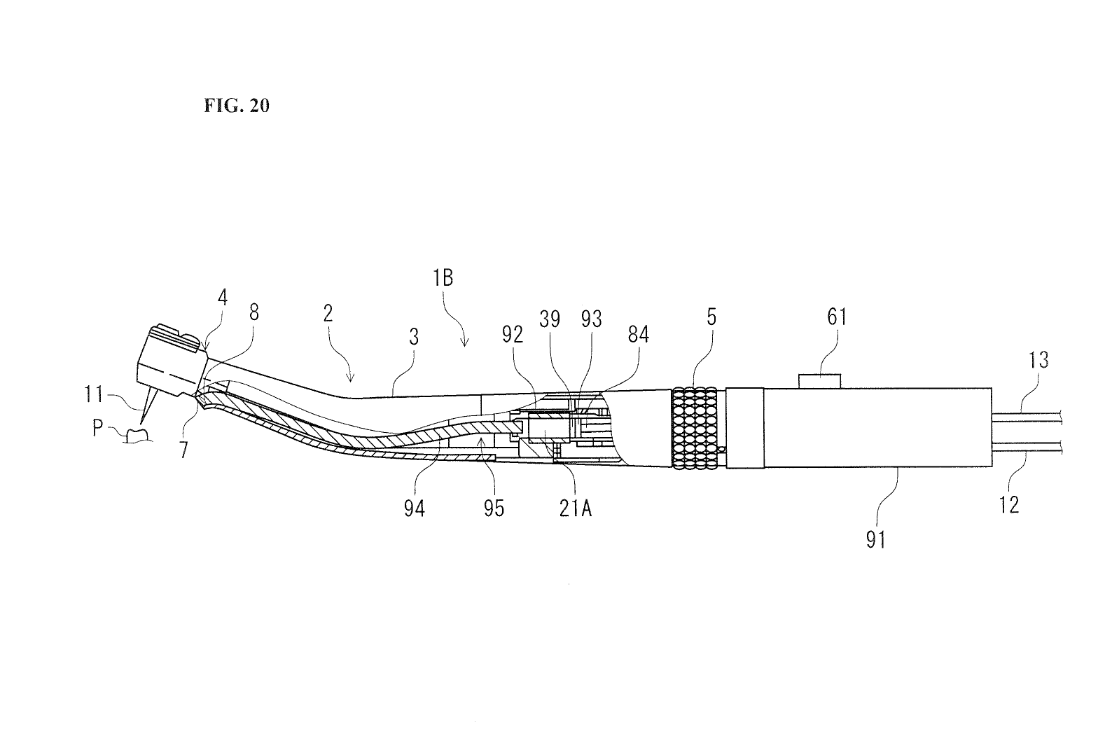

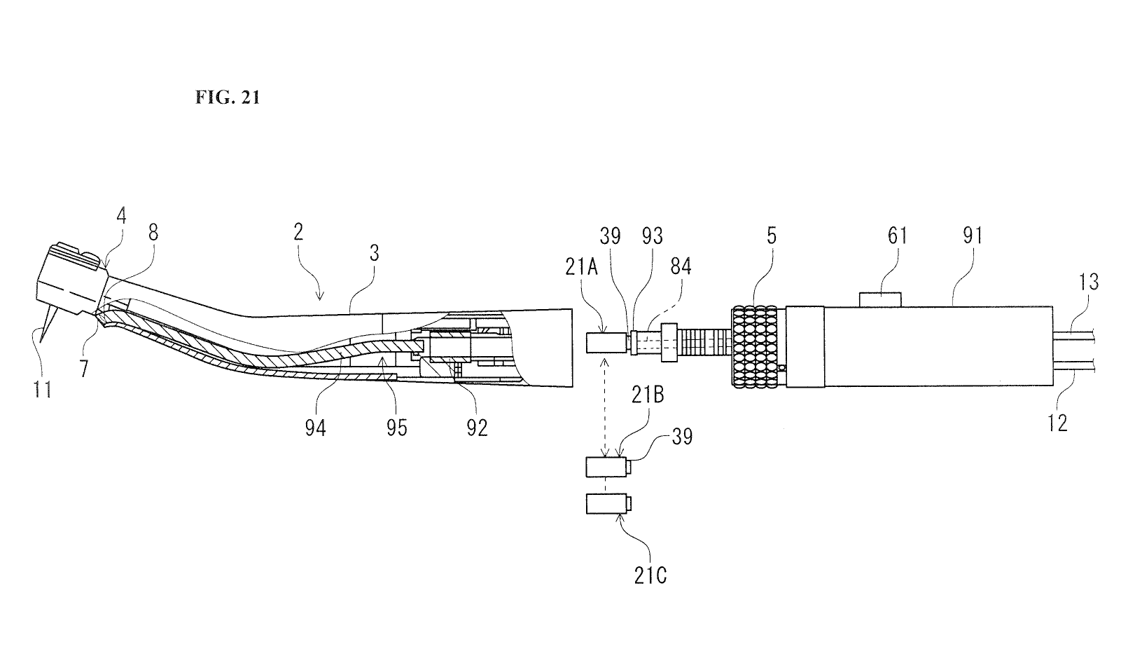

Subsequently, a second embodiment of the present invention will be described with reference to FIGS. 20 and 21.

A dental treatment implement with an anomaly site detection function of the second embodiment is applied to an air-turbine-type handpiece which is a kind of dental treatment instrument.

In the air-turbine handpiece 1B with an anomaly site detection function of the second embodiment (hereinafter, referred to as “handpiece 1B of the second embodiment”), the same elements as in the handpiece 1 of the first embodiment are denoted by the same reference numerals.

As Illustrated in FIGS. 20 and 21, the handpiece 1B of the second embodiment includes: a handpiece body 2 which includes a grip part 3 having a head part 4, where a cutting tool 11 for treating an affected area P is detachably mounted, on the distal end side and a coupling part 5 which is formed in a substantially cylindrical shape and detachably coupled to the grip part 3; and a dental tube part 91 which is detachably mounted at the rear end of the coupling part 5 in the handpiece body 2.

In this arrangement, the distal end of the coupling part 5 is inserted into the central portion of the inside of the grip part 3 and any one (for example, a first color camera module 21A) of first to third color camera modules 21A, 21B, and 21C constituting a color camera module group 21 is detachably arranged in the same manner as in the first embodiment, where the first to third color cameras, modules 21A, 21B, and 21C have detachable compatibility to the holder 92 provided at a fixed place at the distal end of the coupling part

Moreover, each of the first to third color camera modules 21A, 21B, and 21C is configured so as to be attachable/detachable to/from the holder 92 and the receiving connector 93 by attaching or detaching the mounting connector 39 provided in the rear end portion of each of the first to third color camera modules 21A, 21B, and 21C to or from the receiving connector 33 provided, on the holder 92 side.

The receiving connector 93 is connected to one end of a camera cable 84 housing a signal cable and a light emitting element cable, and the camera cable 84 is extended so as to be connected to the lighting operation unit 61, a signal output cable 12 connected to the dental tube part 91, and a light emitting element driving cable 13 in the same manner as in the above.

In the inside of the grip part 3, similarly to the first embodiment, there is arranged a light emission/image pick-up optical system 35 including: an image pick-up window 7, which is formed of, for example, transparent glass material or transparent synthetic resin material, arranged in the position near the head, part 4 and toward the cutting site of the cutting tool 11; an objective lens B arranged inside the image pick-up window 7; and a rod fiber 34 which is a light guide member with a light incident end facing the near area of the objective lens 8 and a light emitting end facing the color camera module 21.

According to the handpiece 1B of the second embodiment, one of the first to third color camera module 21A, 21B, and 21C is selectively mounted on the holder 92 provided on the distal end side of the coupling part 5 on the basis of the configuration including the air-turbine-type handpiece body 2 to perform various anomaly site detection operations for a row of teeth similar to those described above, thereby also enabling the same advantageous effects to be provided. Moreover, similarly to the above, the handpiece 1B also enables efficient implementation of MI in treatment of a dental caries site or the like and efficient implementation of PMTC in brushing guidance and treatment of a row of teeth.

In addition, also in the case of the handpiece 1B of the second embodiment, it is possible to employ a configuration in which the first to third color camera module units 81A, 81B, and 81C similar to those illustrated in FIG. 19 are used and selectively mounted, in the opening 4a provided in the position near the head part 4 so as to achieve the same advantageous effects in the same manner as described above.

Moreover, the handpiece 1 of the first embodiment, the handpiece 1A of the variation, and the handpiece 1B of the second, embodiment described above are able to provide an excellent detection function also regarding an anomaly site such as a softened dentine part, dental tartar, a defect, or a crack in the oral cavity, in addition to the above cases.

The configuration related to the anomaly site detection function in the dental treatment implement of the present invention is also applicable to other types of dental treatment implements such as a laser handpiece, a scaler, and a three-way syringe, in addition to the above cases.

### DESCRIPTION OF REFERENCE NUMERALS

1 Micromotor handpiece with anomaly site detection function
1A Micromotor handpiece with anomaly site detection function
1B Air-turbine handpiece with anomaly site detection function
2 Handpiece body
3 Grip part
4 Head part
4a Opening
5 Coupling part
6 Micromotor
7 Image pick-up window
8 Objective lens
9 Rod fiber
10 Light emission/image pick-up optical system
11 Cutting tool
12 Signal output cable
13 Light emitting element driving cable
14 Stainless-steel cylindrical body
21 Color camera module group
21A Color camera module
21B Color camera module
21C Color camera module
22 Camera head part
24A Light emitting element
24B Light emitting element
24B1 Light emitting element
24C Three-primary-color light emitting element
24C1 Light emitting element chip
24C2 Light emitting element chip
24C3 Light emitting element chip
25 Light emitting element cable
27A Light source unit
27B Light source unit
27C Light source unit
28 Holder
29 Filter
29a Through-hole
30 Filter part
31 Camera unit
32 Support cylinder
33 Condenser lens unit
34 Image pick-up unit
35 Cover member
36 Signal cable
37 Support substrate
38 Color image sensor
38a Sensor substrate
33 Mounting connector
40 Receiving connector
42 Rod fiber
61 Lighting operation unit
62 Switch
63 Changeover switch
64 Full lighting switch
65 Switch
71 Control unit
72 Controller
73 Light emitting element power supply unit
74 Image signal receiving unit
75 Color image generation unit
76 Image storage unit
77 Color image display unit
81A Color camera module unit
81B Color camera module unit
81C Color camera, module unit
82 Cylinder-like body
83 Mounting connector
84 Camera cable
91 Dental tube part
92 Holder
93 Receiving connector
94 Rod fiber
95 Light emission/image pick-up optical system
R1 Resistor
R2 Resistor
R3 Resistor
R4 Resistor
R5 Resistor
R6 Resistor
TR1 Lighting control transistor
TR2 Lighting control transistor
TR3 Lighting control transistor
VR1 Current adjusting variable resistor
VR2 Current adjusting variable resistor
VR3 Current adjusting variable resistor
P Affected area

1. A dental treatment implement with an anomaly site detection function comprising:
a dental treatment implement for treating an anomaly site in an oral cavity;
a color camera module group including a plurality of color camera modules, wherein each color camera module is constructed by integrating a light emission means including a light source unit for at least emitting excitation light for producing fluorescent light at an anomaly site in the oral cavity and an image pick-up means for picking up an image of the anomaly site and surrounding sites in the oral cavity as a color image, wherein the wavelengths of the excitation light emitted from the light source units are different from each other, and wherein one of the color camera modules is detachably mounted in a specific position on the denial treatment implement; and
a light emission/image pick-up optical system for radiating the excitation light from the light source unit of one of the color camera modules mounted in the specific position toward the anomaly site and guiding the fluorescent light produced at the anomaly site to the image pick-up means of the color camera module.

2. A dental treatment implement with an anomaly site detection function comprising:
a dental treatment implement for treating an anomaly site in an oral cavity;
a color camera module group including; a first color camera module constructed by integrating a first light emission means including a light source unit for emitting monochromatic excitation light for producing fluorescent light at an anomaly site in the oral cavity and an image pick-up means for picking up an image of the anomaly site and surrounding sites in the oral cavity as a color image; a second color camera module constructed by integrating a second light emission means formed by combining a light source unit for emitting monochromatic excitation light for producing fluorescent light at the anomaly site in the oral cavity and a light source unit for emitting illumination light for illuminating the oral cavity in such a way as to enable changeover between the light source units or simultaneous emission thereof and an image pick-up means for picking up an image of the anomaly site and surrounding sites in the oral cavity as a color image; and a third color camera module constructed by integrating a third light emission means including a light source unit for selectively emitting excitation light of three primary colors of red, green, and blue for producing fluorescent light at the anomaly site in the oral cavity and an image pick-up means for picking up an image of the anomaly site and surrounding sites in the oral cavity as a color image, wherein the wavelengths of the excitation light emitted from the light source units of the first to third color camera modules are different from each other and wherein one of the first to third color camera modules is detachably mounted in a specific position able to be exposed to the outside or in a specific position enabling operation from the outside by disassembly of the dental treatment implement; and
a light emission/image pick-up optical system for radiating the excitation light from the light source unit of one of the color camera modules mounted in the specific position toward the anomaly site and guiding the fluorescent light produced at the anomaly site to the image pick-up means of the color camera module.

3. A dental treatment implement with an anomaly site detection function comprising:
a dental treatment implement for treating an anomaly site in an oral cavity;
a color camera module group including: a first color camera module constructed by integrating a first light emission means including a light source unit for emitting blue, green, or red excitation light for producing fluorescent light at an anomaly site in the oral cavity and an image pick-up means for picking up an image of the anomaly site and surrounding sites in the oral cavity as a color image; a second color camera module constructed by integrating a second light emission means formed by combining a light source unit for emitting blue, green, or red excitation light for producing fluorescent light at the anomaly site in the oral cavity and a light source unit for emitting illumination light for illuminating the oral cavity in such a way as to enable changeover between the light source units or simultaneous emission thereof and an image pick-up means for picking up an image of the anomaly site and surrounding sites in the oral cavity as a color image; and a third color camera module constructed by integrating a third light emission means including a light source unit for selectively emitting excitation light of three primary colors of red, green, and blue for producing fluorescent light at the anomaly site in the oral cavity and an image pick-up means for picking up an image of the anomaly site and surrounding sites in the oral cavity as a color image, wherein one of the first to third color camera modules is detachably mounted in a specific position able to be exposed to the outside or in a specific position enabling operation from the outside by disassembly of the dental treatment implement; and
a light emission/image pick-up optical system for radiating the excitation light from the light source unit of one of the color camera modules mounted in the specific position toward the anomaly site and guiding the fluorescent light produced at the anomaly site to the image pick-up means of the color camera module.

4. The dental treatment implement with the anomaly site detection function according to claim 1, wherein the dental treatment implement is an angle-type or straight-type micromotor handpiece or an angle-type or straight-type air-turbine handpiece, wherein the color camera module is arranged in a position able to be exposed to the outside by disassembly in the inside of the hand piece body, and wherein the light emission/image pick-up optical system is arranged between the position near the head part where the cutting tool in the handpiece body is arranged and the color camera module.

5. The dental treatment implement with the anomaly site detection function according to claim 1, wherein the dental treatment implement is an angle-type or straight-type micromotor handpiece or an angle-type or straight-type air-turbine handpiece and wherein the color camera module and the light emission/image pick-up optical system are integrated and are formed as a color camera module unit detachably in the position near the head part where a cutting tool in the handpiece body is arranged.

6. The dental treatment implement with the anomaly site detection function according to claim 1, wherein the color camera module is detachably provided with a filter for cutting the wavelength components of the excitation light among lights incident onto the image pick-up means.

7. The dental treatment implement with the anomaly site detection function according to claim 2, wherein the dental treatment implement is an angle-type or straight-type micromotor handpiece or an angle-type or straight-type air-turbine handpiece, wherein the color camera module is arranged in a position able to be exposed to the outside by disassembly in the inside of the handpiece body, and wherein the light emission/image pick-up optical system is arranged between the position near the head part where the cutting tool in the handpiece body is arranged and the color camera module.

8. The dental treatment implement with the anomaly site detection function according to claim 3, wherein the dental treatment implement is an angle-type or straight-type micromotor handpiece or an angle-type or straight-type air-turbine handpiece, wherein the color camera module is arranged in a position able to be exposed to the outside by disassembly in the inside of the handpiece body, and wherein the light emission/image pick-up optical system is arranged between the position near the head part where the cutting tool in the handpiece body is arranged and the color camera module.

9. The dental treatment implement with the anomaly site detection function according to claim 2, wherein the dental treatment implement is an angle-type or straight-type micromotor handpiece or an angle-type or straight-type air-turbine handpiece and wherein the color camera module and the light emission/image pick-up optical system are integrated and are formed as a color camera module unit detachably in the position near the head part where a cutting tool in the handpiece body is arranged.

10. The dental treatment implement with the anomaly site detection function according to claim 3, wherein the dental treatment implement is an angle-type or straight-type micromotor handpiece or an angle-type or straight-type air-turbine handpiece and wherein the color camera module and the light emission/image pick-up optical system are integrated and are formed as a color camera module unit detachably in the position near the head part where a cutting tool in the handpiece body is arranged.

11. The dental treatment implement with the anomaly site detection function according to claim 2, wherein the color camera module is detachably provided with a filter for cutting the wavelength components of the excitation light among lights incident onto the image pick-up means.

12. The denial treatment implement with the anomaly site detection function according to claim 3, wherein the color camera module is detachably provided with a filter for cutting the wavelength components of the excitation light among lights incident onto the image pick-up means.

13. The dental treatment implement with the anomaly site detection function according to claim 4, wherein the color camera module is detachably provided with a filter for cutting the wavelength components of the excitation light among lights incident onto the image pick-up means.

14. The dental treatment implement with the anomaly site detection function according to claim 5, wherein the color camera module is detachably provided with a filter for cutting the wavelength components of the excitation light among lights incident onto the image pick-up means.

15. The dental treatment implement with the anomaly site detection function according to claim 7, wherein the color camera module is detachably provided with a filter for cutting the wavelength components of the excitation light among lights incident onto the image pick-up means.

16. The dental treatment implement with the anomaly site detection function according to claim 8, wherein the color camera module is detachably provided with a filter for cutting the wavelength components of the excitation light among lights incident onto the image pick-up means.

17. The dental treatment implement with the anomaly site detection function according to claim 9, wherein the color camera module is detachably provided with a filter for cutting the wavelength components of the excitation light among lights incident onto the image pick-up means.

18. The dental treatment implement with the anomaly site detection function according to claim 10, wherein the color camera module is detachably provided with a filter for cutting the wavelength components of the excitation light among lights incident onto the image pick-up means.
